(19) United States
(12) Patent Application Publication
(10) Pub. No.: US 2013/0123629 A1
(43) Pub. Date: May 16, 2013

(54) METHOD AND APPARATUS FOR MEASURING THE THICKNESS OF ADIPOSE TISSUE

(71) Applicants: Avner Rosenberg, Bet Shearim (IL); Genady Nahshon, Binyamina (IL); Edward Kantarovich, Rehovot (IL)

(72) Inventors: Avner Rosenberg, Bet Shearim (IL); Genady Nahshon, Binyamina (IL); Edward Kantarovich, Rehovot (IL)

(21) Appl. No.: 13/812,014

(22) PCT Filed: Jul. 13, 2011
(86) PCT No.: PCT/IL11/00555

§ 371 Date: Jan. 24, 2013

Related U.S. Application Data
(60) Provisional application No. 61/367,431, filed on Jul. 25, 2010.

(51) Int. Cl. A61B 8/08

(52) U.S. Cl. CPC A61B 8/0858
USPC 600/442

(57) ABSTRACT

Provided are body fat measuring techniques employed to date, usually applying a certain level of force to the tissue causing narrowing of the adipose tissue layer at the time of measuring. This creates a bias in the adipose layer thickness measurement results that is not accounted for when employing these methods. Provided is a current apparatus and method offering a solution for accounting for this bias thus improving the accuracy of body fat measurements.

### TECHNICAL FIELD

The current method and apparatus relate to the field of devices for measuring thickness of tissue and more specifically to devices for measuring the thickness of adipose tissue.

### BACKGROUND

Obesity is a condition in which abnormal or excessive fat accumulation in adipose tissue impairs health. With all the risks associated with carrying too much body fat, there has been a growing awareness of the benefit to one's health to maintaining a healthy weight and staying within healthy Body Mass Index (BMI) ranges. Measuring one's body fat percentage as part of maintaining a healthy body weight has become prevalent.

Additionally, cosmetic body shaping treatments, also termed body contouring treatments, commonly involve employing complex devices and numerous methods of treatments to reduce body adipose tissue. These devices and treatments include application of various forms of heating energy, mechanical energy and similar. In such treatments it would be useful to obtain accurate information regarding the thickness of the adipose tissue in general and specifically of the adipose tissue in the area being treated.

Many methods of assessing a person's body fat and lean mass have been developed. The most common methods include underwater or hydrostatic weighing, skin fold thickness measurements (caliper), bioelectrical impedance and BMI calculation based on a subject's height and weight.

Some techniques, such as that described in US Patent Application Publications No. 2003/0018257 and No. 2009/0270728 employ ultrasound to measure fat tissue thickness, relying on the varying intensity and/or reflection time of the beams reflected from the various tissue layers. US Patent Application Publication No. 2003/0018257 limits the frequency of the emitted ultrasound beams to above 10 MHz. This technique relies on the inherent density of the various tissue layers to differentiate between them and assess their thickness.

Other techniques, such as that described in US Patent Application Publication No. 2010/0036246 employs ultrasound image analysis techniques to determine the type and thickness of a target tissue.

The technique described by U.S. Pat. No. 5,941,825 discloses measuring body fat from two different locations on the surface of the skin to correct for the parallax error resulting from ultrasound beam emission into the tissue in an angle other than orthogonal.

### SUMMARY

The body fat measuring techniques employed to date, as known to the authors of this disclosure, apply a certain level of force to the tissue causing narrowing of the adipose tissue layer at the time of measuring. This creates a bias in the adipose layer thickness measurement results that is not accounted for when employing these methods. The current apparatus and method offer a solution for accounting for this bias thus improving the accuracy of body fat measurements.

There is thus provided, in accordance with an exemplary embodiment of the current method and apparatus a method of employing an ultrasound transducer for measuring adipose tissue thickness and accounting for a certain level of force of coupling of an applicator to the skin, effecting narrowing of the tissue layers being measured.

In accordance with another exemplary embodiment of the present method and apparatus, there is also provided an applicator including one or more ultrasound transducers and a resilient spacer employing a method of measuring an adipose tissue thickness and accounting for a certain level of force of coupling of the applicator to the skin, effecting narrowing of the tissue layers being measured.

In accordance with yet another exemplary embodiment of the present method and apparatus, there is also provided an applicator including one or more ultrasound transducers and one or more RF electrodes employing a method of measuring an adipose tissue thickness and accounting for a certain level of force of coupling of the applicator to the skin, effecting narrowing of the tissue layers being measured, employing reflected ultrasound beam signals and adipose tissue RF impedance measurement.

In accordance with still another exemplary embodiment of the present method and apparatus, there is also provided an apparatus including one or more RF electrodes divided into one or more external segments and one or more internal segments driven at the same potential and measuring separately the current through each segment to obtain differentiation between the current flowing through skin tissue and the current flowing through fat tissue.

In accordance with still another exemplary embodiment of the present method and apparatus, there is also provided a method of measuring water content of adipose tissue employing reflected ultrasound beam signals and RF electrodes to measure adipose tissue conductivity.

### BRIEF DESCRIPTION OF THE DRAWINGS

The present method and apparatus will be understood and appreciated from the following detailed description, taken in conjunction with the drawings in which:

FIGS. 1A and 1B are simplified views of an exemplary embodiment of the current method and apparatus;

FIGS. 2A, 2B, 2C and 2D are simplified illustrations of an exemplary method of implementation of the embodiment of FIGS. 1C and 1D in accordance with the current method and apparatus;

FIGS. 3A and 3B are simplified illustrations of another embodiment of the current method and apparatus;

FIG. 4 is a simplified illustration of an exemplary method of implementation of the embodiment of FIGS. 3A and 3B in accordance with the current method and apparatus;

FIGS. 5A and 5B are simplified illustrations of received signals of portions of an ultrasound beam in accordance with another embodiment of the current method and apparatus;

FIG. 6 is a simplified illustration of an embodiment of an adipose tissue thickness measuring device applicator in accordance with the current method and apparatus;

FIGS. 7A, 7B, 7C, 7D, 7E, 7F, 7G, 7H and 7I are simplified plan-view and cross-sectional view illustrations of various examples of configurations and exemplary embodiments of the apparatus of FIG. 6.

FIG. 8 is a graph illustrating the dependence of adipose tissue impedance on a force effecting narrowing of the adipose tissue.

FIGS. 9A, 9B, 9C and 9D are simplified illustrations of an exemplary method of implementation of the embodiment of FIG. 6 in accordance with the current method and apparatus;

FIG. 10 is a simplified illustration of the effect of RF frequency on tissue conductivity or impedance of tissue layers interposed between RF electrodes in accordance with the current method and apparatus; and

FIG. 11 is a graph illustrating the frequency dependence of the conductivity or impedance of adipose, skin and muscle tissues.

### DETAILED DESCRIPTION

For the purpose of this disclosure the terms “fat”, “fat tissue” or “adipose tissue” as used in the present disclosure have the same meaning and are used interchangeably throughout the disclosure. It should also be understood that the apparatuses, processes and treatments disclosed below may also be applicable to other types of tissue.

The term “a certain level of force” as used in the present disclosure means a level of force which may be known, previously recorded, predetermined or arbitrary, determined in real time or arrived at empirically.

The term “water” as used in the present disclosure means any electrically conductive naturally or artificially occurring fluid in and around tissue such as edema, exudate, transudate, tumescent solution or fluid such as a solution of sterile dilute salt water, adrenaline, lidocaine, anesthetic material or other ingredients injected into the adipose tissue during a cosmetic body contouring procedure.

The term “treatment” as used in the present disclosure means an aesthetic or cosmetic procedure of coupling to the tissue or skin energy affecting the tissue or skin appearance.

The term “narrowing” as it relates to “fat”, “fat tissue”, or “adipose tissue” and used in the present disclosure means narrowing of the “fat”, “fat tissue” or “adipose tissue” layer thickness as a result of an applied level of force exerting pressure on the tissue.

The terms “emitting” and “radiating” as related to ultrasound beams or ultrasound beam pulses are used interchangeably in the present disclosure and mean generation of any type of ultrasound energy from an ultrasound transducer.

### Adipose Tissue Thickness Measurement Employing an Ultrasound Transducer

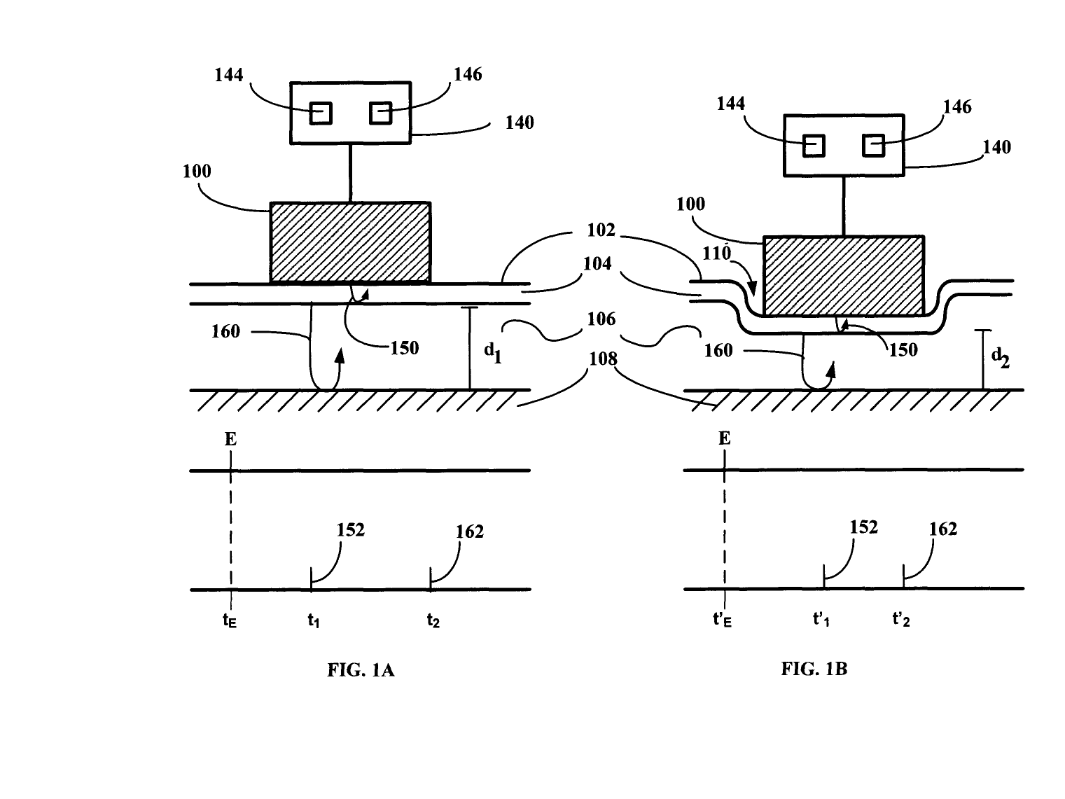

Reference is made to FIGS. 1A and 1B, which are simplified views of exemplary embodiments of the current method and apparatus. FIG. 1A illustrates an ultrasound transducer 100, which communicates with a control unit 140 including, among others, a source of power 144, and an ultrasound driver 146 coupled to a surface 102 of skin 104. In the exemplary embodiment of FIGS. 1A and 1B and in accordance with the current method and apparatus, when activated, transducer 100 emits ultrasound beams in pulse form, which propagate throughout the tissue. The ultrasound beam pulses may be emitted concurrently or consecutively. Portions of the emitted beams are reflected from tissue interfaces (surfaces disposed between adjacent tissue layers having different acoustic indexes).

Separation between the transmitted ultrasound beams and the received portions thereof may be achieved in the time domain by emitting the beams in pulse form, or in the frequency domain by varying the frequency within a band to isolate the reflected pulse as will be further described in detail below.

In FIG. 1A, for example, a portion of a beam emitted by transducer 100 is reflected from the skin layer 104 and adipose tissue layer 106 interface as indicated by an arrow designated reference numeral 150 and is represented by a received signal 152 received by transducer 100 at (t₁)) measured from time of emission (t_{E}). Another portion of the emitted beam is reflected from a deeper adipose tissue layer 106-muscle layer 108 interface as indicated by arrow designated reference numeral 160 and is represented by signal 162 received by transducer 100 at (t₂)) measured from time of emission (t_{E}). The thickness (d₁) of fat tissue layer 106 may then be calculated from the time difference (t₂−t₁) between received reflected beam portion signals 152 and 162 and known velocity of sound in fat tissue.

This technique, which is widely used in the art, is sometimes deficient in that it does not account for the narrowing of the adipose tissue layer effected by the force of coupling of the measuring device applicator (in this case, an ultrasound transducer). This bias resulting from this unavoidable narrowing may be highly significant in soft fat layers having physical properties closer to those of fluids than to those of solids.

FIG. 1B illustrates the above described bias effect. When applied to a single selected location on the skin at a certain level of force, transducer 100 presses upon skin layer 104 creating a depression 110. As a result, adipose tissue layer 106, which is much more fluid in nature than skin layer 104 and muscle layer 108 escapes from the area under transducer 100 and flows to the sides, narrowing fat layer thickness from (d₁) to (d₂) and shortening the propagation and reflection time of beam portion 160 from (t₂) to (t′₂). The skin layer, being much less fluid in nature than the fat tissue is almost unchanged, so (t′₁) is very close to (t₁).

Alternatively, the calculation may employ only ultrasound beam pulse portion 160 to receive the thickness of fat tissue layer 106 and skin layer 104 combined. In some application this may be a required quantity. Since the thickness of skin in various areas of the human body is well documented, the skin thickness at the sight of measurement may be derived from a lookup table and be subtracted from the combined fat tissue layer thickness and skin to arrive at the thickness of fat tissue layer 106 alone.

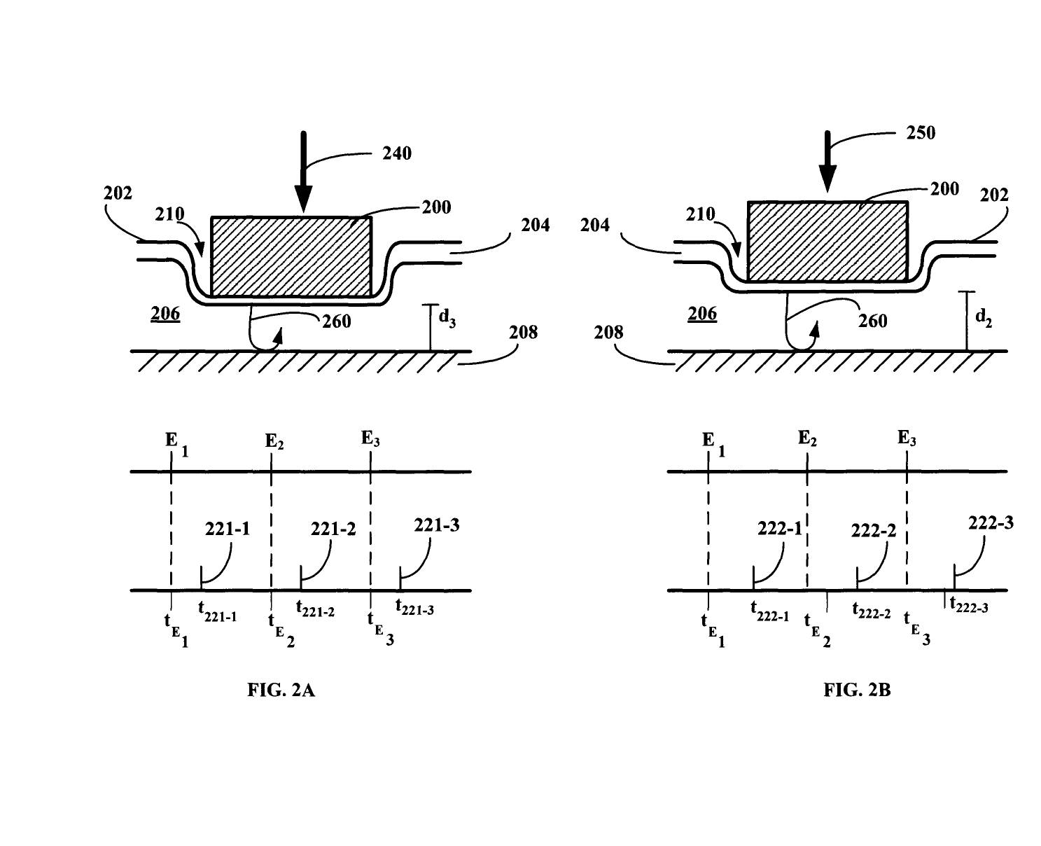

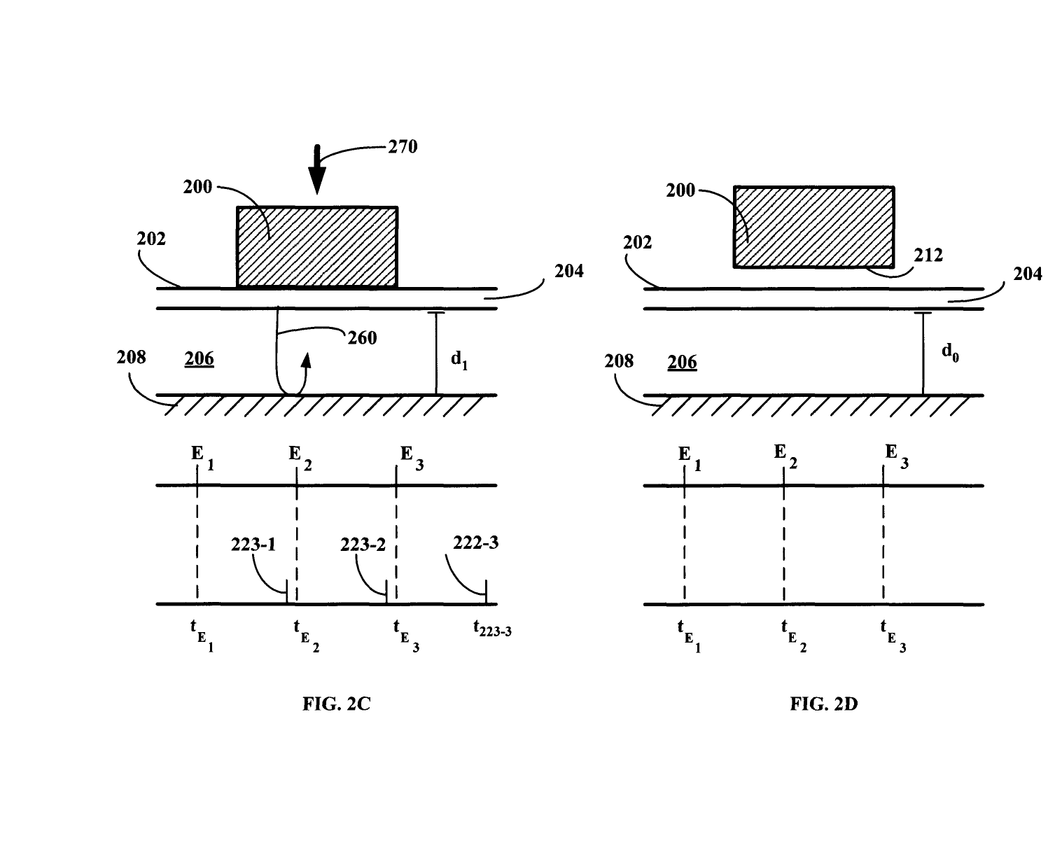

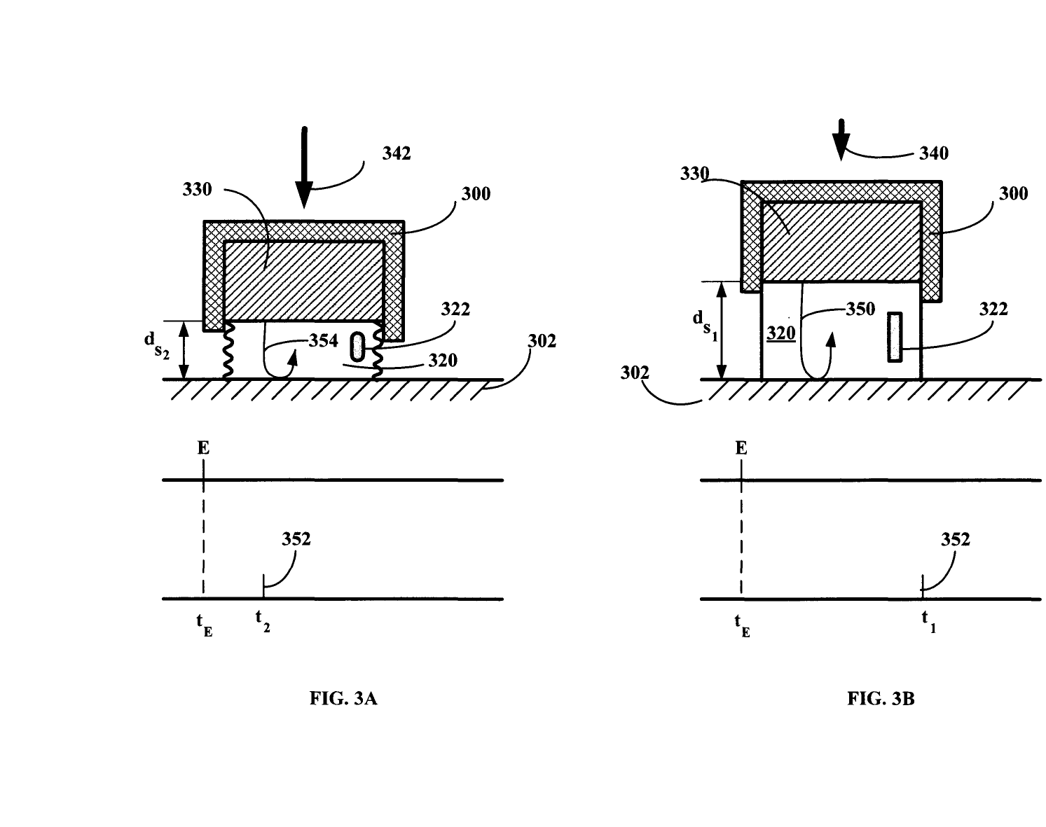

Referring now to FIGS. 2A, 2B, 2C and 2D, which are simplified illustrations of an exemplary method of implementation of the embodiment of FIGS. 1A and 1B in accordance with the current method and apparatus. In FIG. 2A, transducer 200 is coupled to the surface 202 of skin layer 204 at a certain level of force indicated by an arrow 240 creating depression 210 and decreasing fat tissue layer 206 thickness to a thickness (d₃). Transducer 200 is activated to emit ultrasound beams in pulse form at an emission times (t_{E}). The recorded emitted signals are designated by the letter (E). A series of pulse signals 221 of beam pulse portions 260 reflected from fat 206-muscle 208 interface are received and recorded, displaying a time of reception (t₂₂₁₋₁, t₂₂₁₋₂, t₂₂₁₋₃ . . . ).

In FIG. 2B the level of force at which ultrasound transducer 200 is coupled to surface 202 is then gradually reduced, manually or automatically, as indicated by arrow 250 bringing about the reduction in the depth of depression 210 of skin layer 204 and an increase in fat tissue layer 206 thickness to a thickness (d₂). Pulse signals 222 continue to be recorded, now displaying a longer time gap between time of emission (t_{E}) of the emitted pulses (E) and time (t₂₂₂) of received pulse signals 222, for example, (t₂₂₂₋₁>t₂₂₁₋₁), indicating the change in fat tissue layer 206 thickness from (d₃) to a thickness (d₂) affecting changes in the propagation times of reflected portions 260.

In FIG. 2C the process described in FIG. 2B is repeated. The level of force at which ultrasound transducer 200 is coupled to surface 202 is further gradually reduced, manually or automatically, as indicated by arrow 270 to a point of disengagement (end point or disengagement point) of ultrasound transducer 200 emitting surface 212 from skin 204 surface 202. At this end point, which is as close to optimal as possible, transducer 200 is coupled to skin 204 surface 202 at a minimal level of force or, optimally, with no application of force. No noticeable depression 210 exists and measured fat layer thickness (d₁) is as close as possible to true thickness (d₀) which prevails at rest (i.e., with no contact between transducer 200 emitting surface 212 and skin 204 surface 202 as shown as FIG. 2D).

Immediately following the end point (disengagement or zero force point) of FIG. 2C, contact between transducer 200 emitting surface 212 and skin 204 surface 202 is broken, as illustrated in FIG. 2D. At this instance, no reflected pulse signals are received. This implies that the time of reception (t₂₂₃₋₃) of the last recorded pulse signal 223-3 (FIG. 2C) represents the most precise indicator of thickness (d₁) of adipose tissue layer 206. In other words, i.e., thickness (d₁) at time of measurement of pulse 223-3 is closest to true thickness (d₀) without application of pressure to the skin-zero force adipose layer thickness.

In the above description, measurement of the thickness of fat tissue layer 206 may or may not include beam portion 150 (FIGS. 1A and 1B) reflected from the skin 204-fat tissue layer 206 interface. In the embodiment illustrated in FIG. 2, the value of skin layer 204 thickness may be derived from a lookup table (as described hereinabove).

### Adipose Tissue Thickness Measurement Employing an Ultrasound Transducer and a Spacer

Referring now to FIGS. 3A and 3B, which are simplified illustrations of another embodiment of the current method and apparatus. According to the current embodiment, the reflected ultrasound can be used to measure the spacer thickness and deduce from the spacer resilient properties the level of force at which applicator 300 is applied to surface 302. An applicator 300 including a resilient spacer 320 attached to the emitting surface of an ultrasound transducer 330 of the type depicted in FIG. 1 is coupled to a rigid surface 302. Spacer 320 may be made of a resilient material selected from a group consisting of rubber, epoxy and a polymer, or have a resilient structure including a bias element such as a spring and filled with liquid acoustic transmission media. The resilient spacer may be of a known initial thickness and selected to have a known modulus of elasticity or if the resilient force is generated by a bias such as a spring, the spacer may have a known spring constant.

In another embodiment, spacer 320 may also include one or more strain measuring elements (322) such as a strain gauge that communicates with a control unit 140 (FIG. 1).

In yet another embodiment, spacer 320 may be made of a piezoelectric material and be operative to respond to pressure effected by the level of force of applicator 300 coupling to surface 302 and respond to the level of force by producing an electrical signal to control unit 140 (FIG. 1) indicating changes in the level of force.

In still another embodiment, ultrasound transducer 330 itself may be operative to respond to pressure effected by the level of force of applicator 300 coupling to surface 302 and respond to the level of force by producing an electrical signal to control unit 140 (FIG. 1) indicating changes in the level of force.

As shown in FIG. 3A, applicator 300, transducer 330 and attached spacer 320 are coupled to a rigid surface 302 at a certain level of force (N) as indicated by arrow 340. Force (N) may be a force of coupling pressed against the tissue surface exerting pressure at the location where the adipose tissue thickness is being measured. Force (N) may be applied manually by an operator or automatically by an aesthetic treatment applying device. A portion of a beam emitted by transducer 330 through resilient spacer 320 is reflected from rigid surface 302 as indicated by an arrow designated reference numeral 354 and is represented by a signal 352 received after a time period of (t₁) measured from time of emission (t_{E}). The time gap between the transmitted signal (E) and received reflected beam portion 350 signal 352 is used to calculate spacer thickness, spacer strain and force (N).

The procedure described in FIG. 3A above and in FIG. 3B is a calibration stage, which may be performed by the user. Alternatively, the physical properties of the resilient spacer may be predetermined by the composition of the material selected for spacer production. Additionally, the spacer may be calibrated in production and provided pre-calibrated by the manufacturer. The calibration information may be supplied by the manufacturer with the pre-calibrated resilient spacer.

In FIG. 3B, applicator 300, transducer 330 and attached spacer 320 are coupled to rigid surface 302 at a greater certain level of force (N′) so that (N′)<(N) as indicated by an arrow designated the reference numeral 342. A portion of a beam emitted by transducer 330 through resilient spacer 320 is reflected off rigid surface 302 as indicated by an arrow designated reference numeral 354 and is represented by a signal 352 received after a time period of (t₂) measured from time of emission (t_{E}). Time period (t₂) is shorter than time period (t₁) designating the compression of spacer 320 thickness dₛ from (dₛ₁) to (dₛ₂).

The correlation between (dₛ₁) and (dₛ₂) at various levels of force may be employed to calculate the force (N) from the reflected ultrasound as well as the thickness (d) at a zero level of force. The correlation between (dₛ₁) and (dₛ₂) at various levels of force of coupling and the time of reception of their corresponding signals may then be derived empirically, be recorded and arranged in a database such as a lookup table. This data may be also collected for various ultrasound frequencies, various resilient spacers having various thicknesses and various moduli of elasticity, having various acoustic properties and other varying applicable factors. In actuality, this may serve as a spacer calibration process.

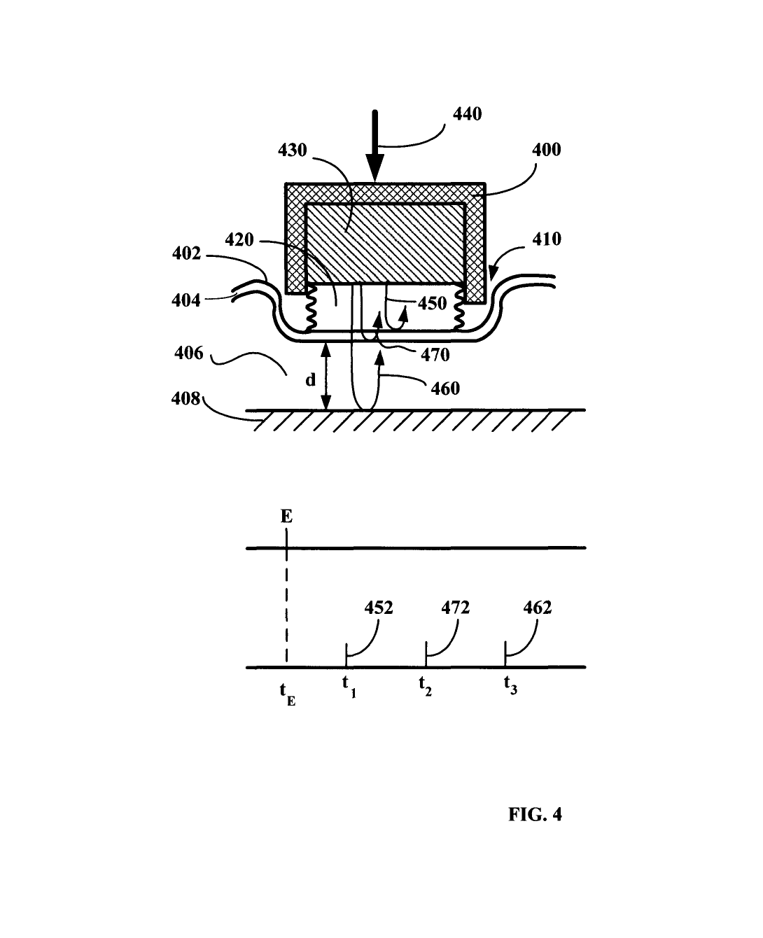

Reference is now made to FIG. 4, which is a simplified illustration of an exemplary method of implementation of the embodiment of the spacer shown in FIGS. 3A and 3B in accordance with the current method and apparatus and in a state of compression similar to that shown in FIG. 3B. An applicator 400 including a transducer 430 and a resilient spacer 420, such as that shown in FIGS. 3A and 3B, are coupled to the surface 402 of skin 404 at a certain level of force indicated by an arrow 440, creating a depression 410 in skin 404, compressing spacer 420 and effecting the narrowing of fat tissue layer 406 thickness to a thickness (d).

A portion of a beam emitted by transducer 430 through now compressed resilient spacer 420 is reflected from spacer 420-surface 402 of skin 404 interface as indicated by an arrow designated reference numeral 450 and is represented by a signal 452 received after a time period of (t₁) measured from time of emission (t_{E}). Another portion of the emitted beam is reflected from a deeper adipose tissue layer 406-muscle layer 408 interface as indicated by arrow designated reference numeral 460 and is represented by signal 462 received at (t₃)) measured from time of emission (t_{E}).

Another beam portion 470 is reflected from the skin 404-fat 406 interface because of acoustical impedance mismatch and is represented by signal 472.

The process described hereinabove enables the measuring of the fat layer thickness vs. level of force of coupling. During the measurement session, the caregiver or an automatic system may apply varying levels of force to the applicator. During this time, the transducer transmits a sequence of pulses, and the reception times of pulses reflected from spacer 420-skin 404, skin 404-fat 406 and fat 406-muscle 408 interfaces are recorded. The pulse signals reflected from spacer 420-skin 404 interface or skin 404-fat 406 interface may be used to deduce the level of force of coupling and the pulse signals reflected from fat 406-muscle 408 interface may be used to deduce fat layer 406 thickness. This method and apparatus may be employed to obtain the value of fat thickness vs. applicator level of force of coupling. This data (i.e., fat thickness and applicator level of force of coupling) may also be used for deriving fat elastic properties and/or to obtain fat layer thickness at a specific level of force, which may be used as a reference for all measurements. Zero force point or disengagement point may also be identified by this measurement, to obtain the value of undisturbed fat tissue thickness.

The acoustical properties of the spacer, specifically the acoustical impedance, may be selected to be close or identical to that of the skin to eliminate skin reflected signal isolating only the skin 404-fat 406 interface reflected signal, or, alternatively, a spacer may be selected with an impedance as close as possible, but different than that of skin so that to sufficiently allow detection of spacer 420-skin 404 interface reflection, so skin thickness may also be measured.

When the acoustic impedance of spacer 420 is selected to match the impedance of the skin, the first reflection signal 470 will be obtained from the skin 404-fat 406 interface. To measure spacer 420 thickness by this reflection 470 one has to assume fixed skin thickness. The acoustic impedance of the spacer 420 can be selected to be slightly different from that of the skin, to generate a reflected signal 450 from the spacer-skin interface. This reflection may be used to measure spacer thickness directly. The difference between spacer and skin impedances can be selected to be at the minimal value required to generate measureable return signal, and not much larger to prevent too much loss at the spacer-skin interface and enable enough power propagation into the deeper fat layer.

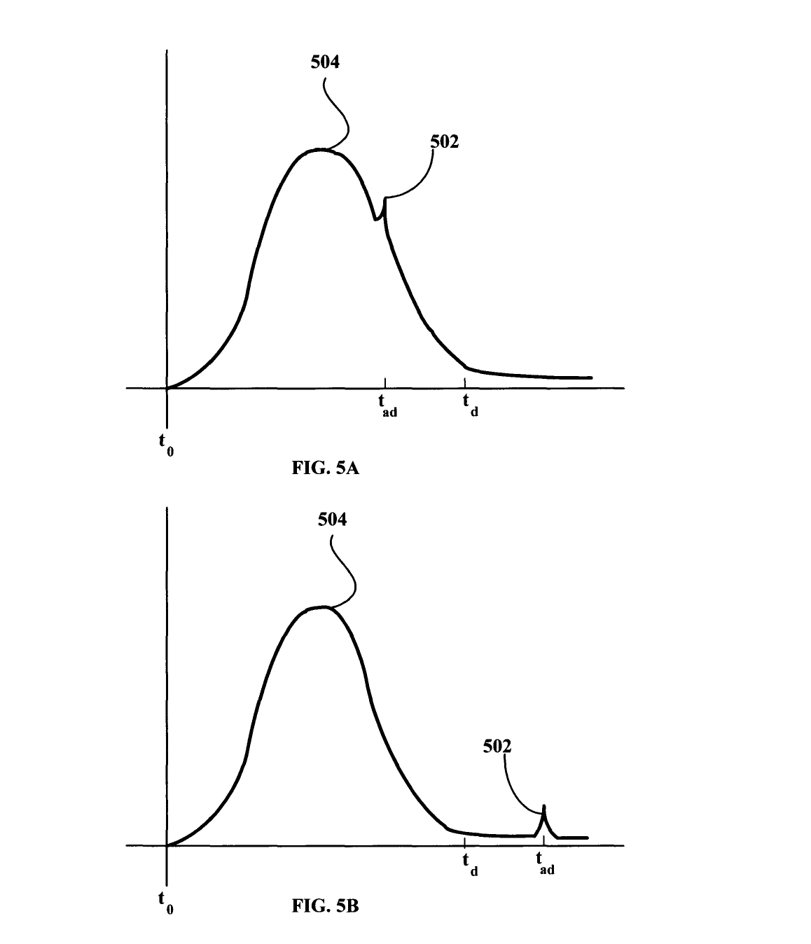

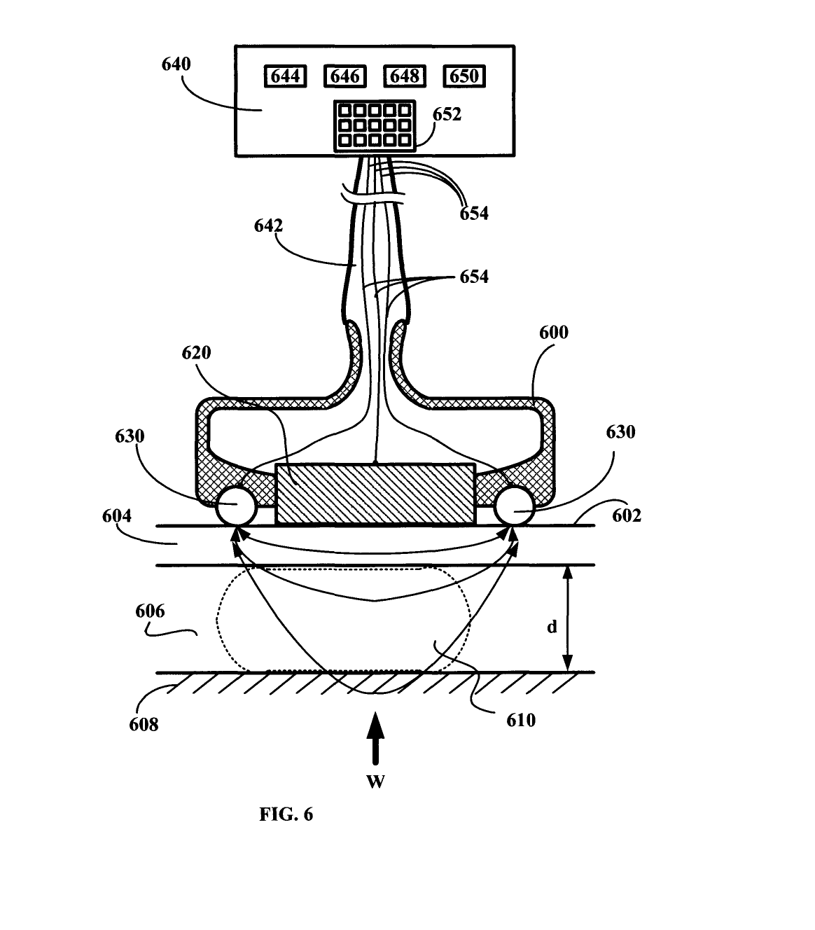

Reference is now made to FIGS. 5A and 5B, which are simplified illustrations of received signals of portions of an ultrasound beam in accordance with another embodiment of the current method and apparatus. In FIG. 5A the time (t_{ad}) of reception of a signal 502 of the portion of the beam reflected from the skin-adipose (or adipose-muscle tissue) tissue interface and measured from time of emission (t_{E}) may be shorter than the decay time (t_{d}) of the transmitted signal, depicted by line 504 and therefore might be partially/fully masked.

In accordance with the current method and apparatus, a spacer, such as that described hereinabove, or a non-resilient spacer, may also be operative to delay beam portion reflections to a point in time beyond transmitted signal decay time (t_{d}).

FIG. 5B, illustrates the effect of adding a spacer having acoustic properties operable to delay reflected beam portions in received signal in accordance with the current method and apparatus. The delayed reflected signals includes all signals of interest, such as spacer-skin, skin-fat and fat muscle interface reflection. Such an acoustic spacer may enable the isolation of a signal 502 reflected from tissue interfaces, and enhance the accuracy of thickness measurement of the desired tissue layer.

A spacer of the type described in FIGS. 3A and 3B may also have an acoustic index matched to that of skin so that to eliminate reflection of a portion of the ultrasound beam from the surface of the skin, such as that indicated by reference numeral 450 (FIG. 4).

Other methods to isolate the pulse signal reflected from the adipose tissue 106-muscle 108 interface (FIG. 1), in accordance with the current method and apparatus, may also employ techniques such as Linear Frequency Modulation (FM).

It is well known in the art that in echo systems, such as an ultrasound echo system, the range resolution is related to the transmitted bandwidth. The transmitted bandwidth is inversely proportional to the pulse width. As described in FIGS. 5A and 5B hereinabove beams in short pulse mode are radiated and reflected. However, instead of using real short pulses, virtual pulses or equivalent to short pulses may be formed by continuous or stepwise transmission of frequencies covering the same bandwidth as the real or virtual pulse. Standard transform techniques may then be employed by computerized processing to transform the results from frequency domain to time domain and isolate the virtual pulse reflected from the adipose tissue from the frequency dependent reflections.

When employing the Linear Frequency Modulation (FM) technique, the transmitted frequency of the radiated pulses is scanned linearly within a frequency band and the returned signal is mixed with the transmitted signal. The resulting frequency difference is directly proportional to the tissue thickness range.

Employing the aforementioned techniques, the following considerations may also be included when selecting the frequency range (or equivalently, pulse length):

a) Since typical sound velocity (v) in tissue is 1500 m/sec, an added fat thickness (d) of, for example, 1 mm of will increase the delay of the return signal by 1.33 microseconds [(d/v)×2=(0.001/1500)×2=1.33 microseconds]. Therefore for a resolution better than 1 mm the pulse front rise time must be of the order of 1 microsecond, which means that the spectral content of the pulse should have a bandwidth above about 200 kHz.

b) Considering the attenuation of the acoustic wave in the fat layer and to prevent excessive loss in reflected signal intensity, it is advisable to use frequencies lower than a few MHz, since attenuation in tissue is proportional to frequency. To reduce attenuation frequency lower then 3 MHz or lower than 1 MHz may be used.

c) Still another consideration in selecting frequency range (or equivalently, pulse length) is avoiding too many details in the reflection. The reflection of interest is that reflected from fat-muscle interface. Hence, it is desirable to weaken reflections from small irregularities in the tissue. Lower frequencies will average these irregularities reflections with no effect on the fat-muscle reflection. In one embodiment employed in accordance with the current method and apparatus, the ultrasound frequency may be scanned between 200 kHz and 2 MHz. In another embodiment, the ultrasound may be transmitted in pulsed mode, pulse signal rise time being between few tens to few hundreds of nanoseconds, more specifically the pulse signal rise time being between 50 nsec to 500 nsec, the pulse signal width being between 0.1 to 10 microseconds. Alternatively, the transducer area may be large enough to generate a broad beam which averages non-uniformities in the fat tissue. Since typical collagen structures within the fat layer are a few mm in size, the transducer radiating aperture width may be selected to be larger than 5 mm, or, more specifically larger than 10 mm.

### Adipose Tissue Thickness Measurement Employing Ultrasound and RF Impedance Measurement

Reference is now made to FIG. 6, which is a simplified illustration of an embodiment of an adipose tissue thickness measuring device applicator in accordance with the current method and apparatus. An adipose tissue thickness measuring device applicator 600 includes one or more ultrasound transducers 620 and one or more RF electrodes 630.

Applicator 600 is connected to a control unit 640, which includes a power source 644. Power source 644 is connected to an ultrasound driver 646 and RF generator 648. Control unit 640 also contains a processor 650 for monitoring impedance and controlling various functions of the system. Processor 650 may also be operative to calculate from the impedance measured between the electrodes the level of narrowing of adipose tissue effected by the coupling of applicator 600 as will be described below.

Control unit 640 may also have an input device, such as a keypad 652 that allows an operator to input to processor 648 selected values of parameters of the measurement and/or treatment, such as the frequency, pulse duration and intensity of the ultrasound and RF energy to be directed to the adipose tissue.

Applicator 600 is connected to control unit 640 via a harness 642 cables 654 to supply power to ultrasound transducer 620 and RF electrodes 630.

Ultrasound transducers 620 and one or more RF electrodes 630 may be coupled at a certain level of force to a surface 602 of a skin layer 604. Alternatively and optionally, all or part of ultrasound transducer 620 may also be operative to operate as an RF electrode or electrodes, by covering its surface with electrically conducting layer or grid which has a low attenuation of ultrasound waves as will be explained in detail below. Alternatively, in a mono-polar configuration, a separate return electrode may be employed. Optionally, ultrasound transducer 620 may also include a resilient or rigid spacer and operate as in the embodiments described in detail hereinabove.

In the current embodiment, RF electrodes 630 are employed to enable measuring of electrical impedance of a tissue segment, mainly adipose tissue layer 606 volume 610, disposed between electrodes 604 as a real time indicator of the coupling force effecting narrowing and affecting measured thickness of adipose tissue layer 606, as will be described in detail hereinbelow.

Electrodes 630 placed, for example, on the surface 602 of skin 604 may be employed to determine the electrical impedance of the adipose tissue volume 610 disposed between electrodes 630 by applying a certain RF voltage between the electrodes and measuring the current between them. The current path in the tissue can be from the electrode, through the skin back to the other electrode, from the skin to the fat and back to the skin and to the other electrode, or in the path electrode-skin-fat-muscle-fat-skin-electrode. The current division between these paths depends on the tissue properties and on the electrodes configuration. At a frequency of about 1 MHz the resistance of the fat is about ten times that of the skin, and the resistance of the muscle is about half that of the skin. The larger the separation between the electrodes, the larger the portion of current flowing in the paths which includes the fat and the muscle.

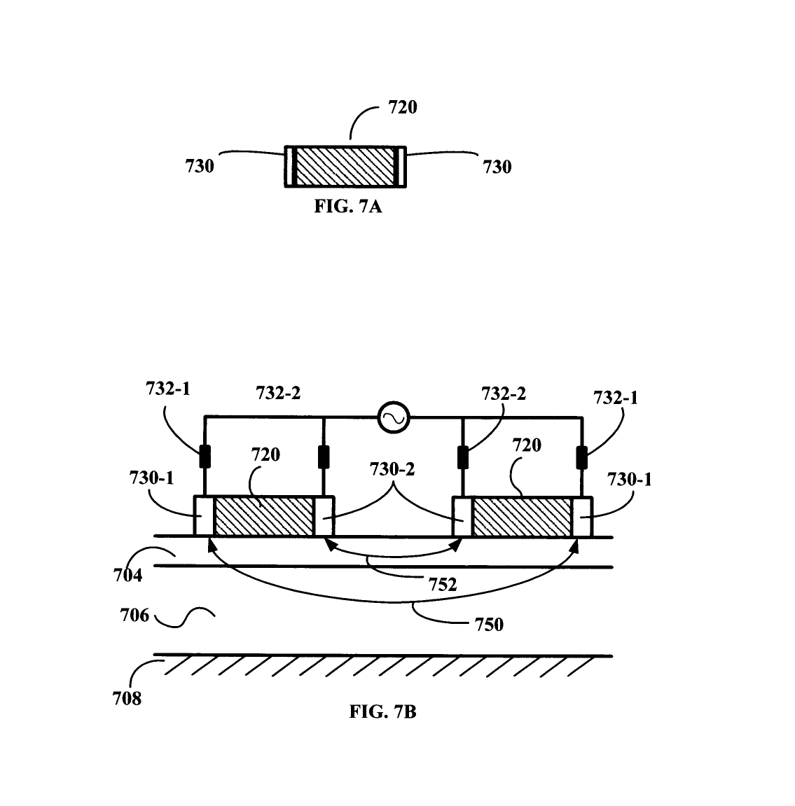

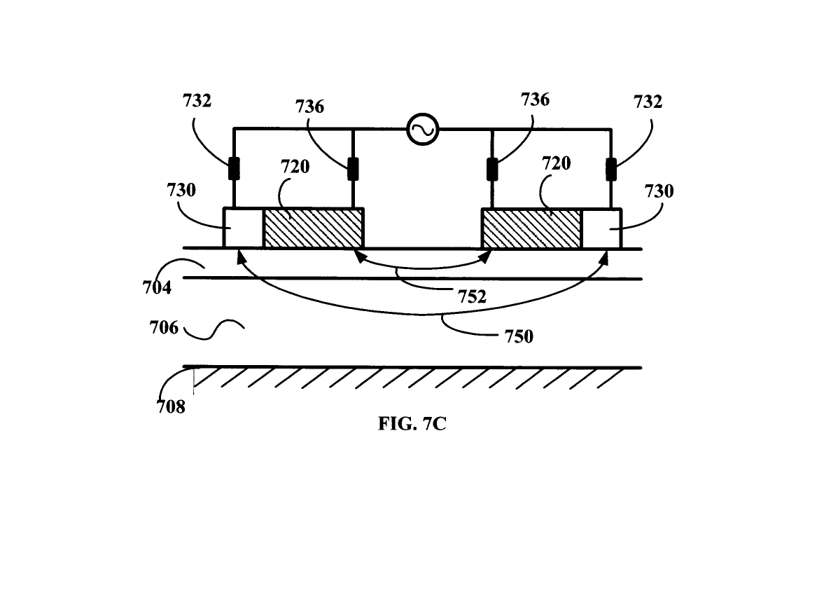

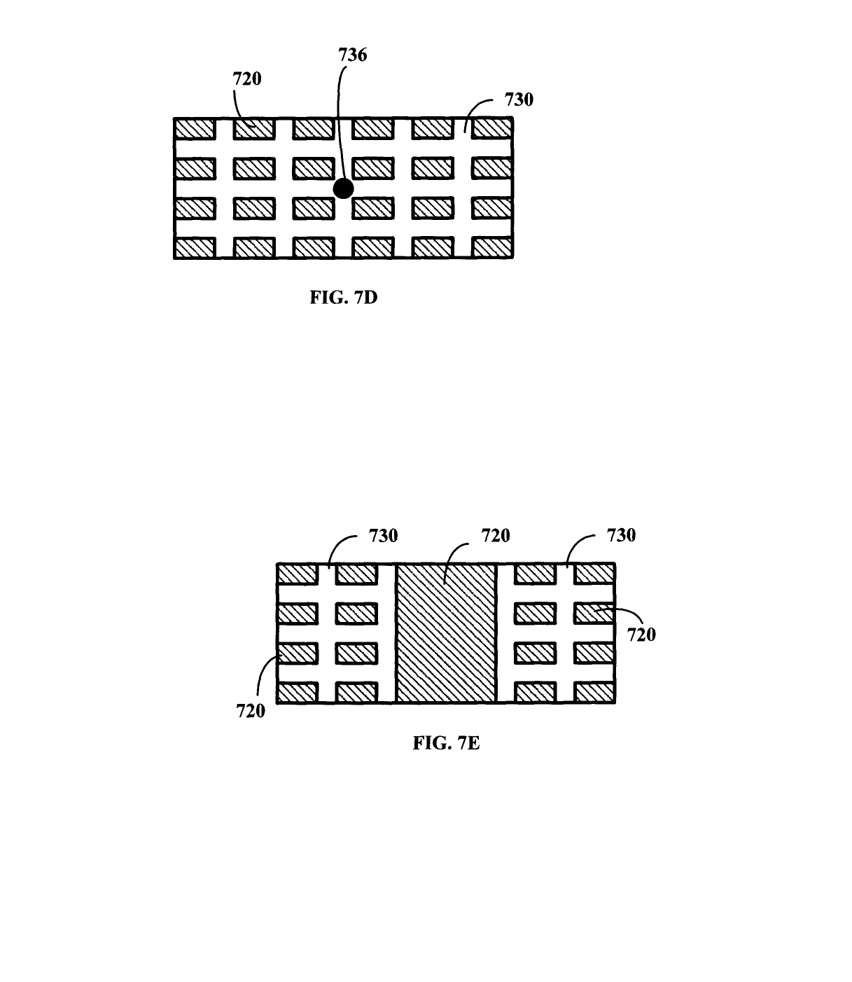

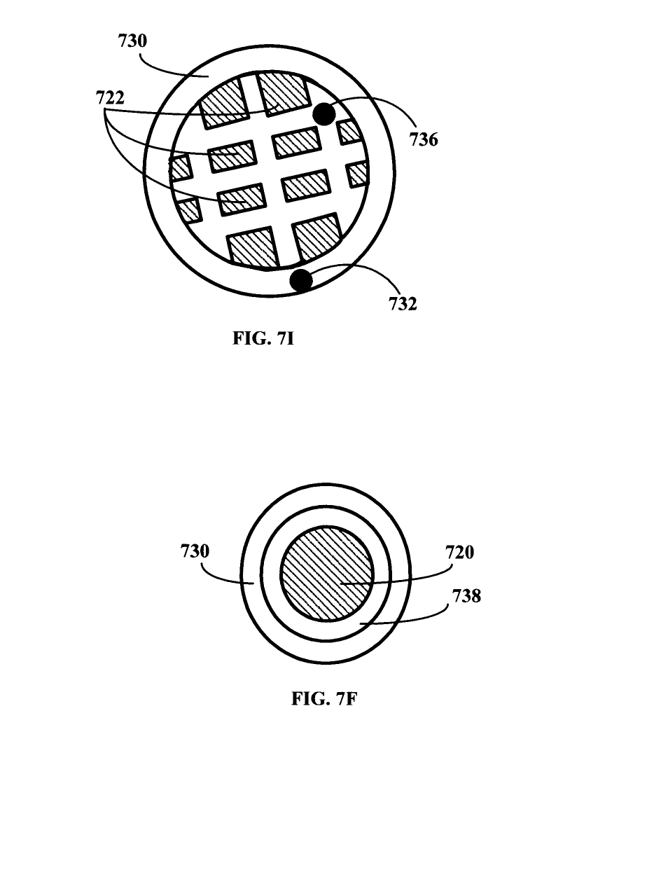

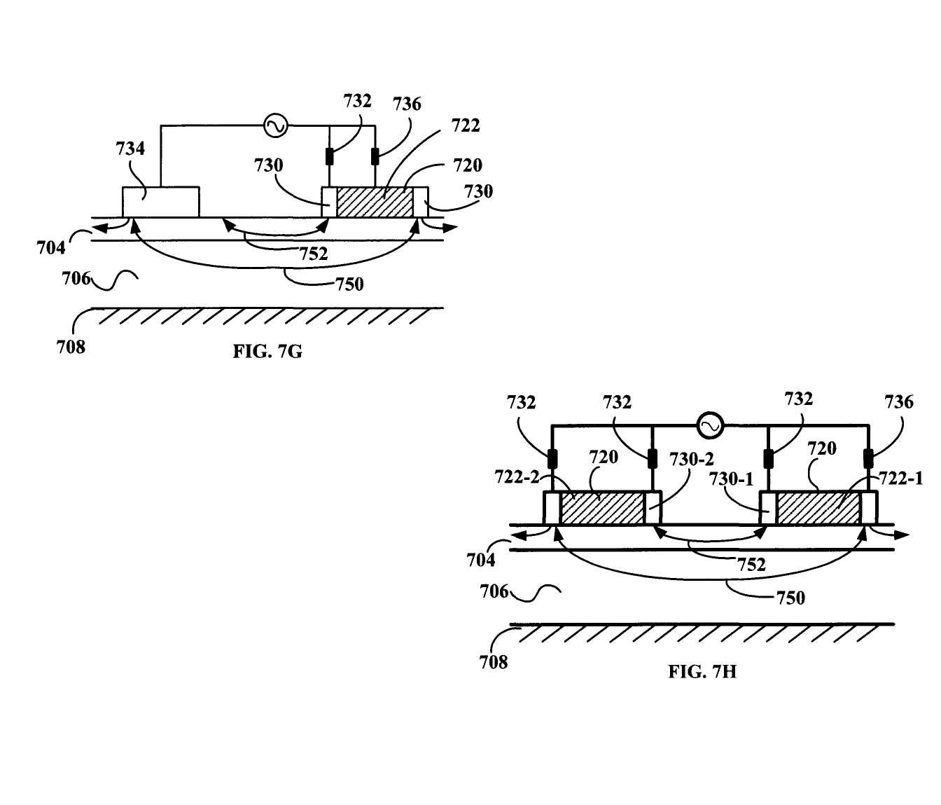

FIGS. 7A, 7B, 7C, 7D, 7E, 7F, 7G, 7H and 7I are simplified illustrations of various examples of configurations and exemplary embodiments of the apparatus of FIG. 6 as viewed from the direction indicated by arrow (W). One or more RF electrodes 730 may be disposed on one or more sides of an ultrasound transducer 720. For example, and as shown in FIG. 7A, which is a simplified illustration of an exemplary embodiment in accordance with the current method and apparatus, one or more RF electrodes 730 are disposed on opposite sides of an ultrasound transducer 720.

In FIG. 7B, which is a cross-sectional view illustration of another exemplary embodiment, one or more RF electrodes 730 are located at any one or more sides of one or more ultrasound transducers 720 such as, for example, that depicted in FIG. 7A. In FIG. 7B, electrodes 730 are equipotential. Current sensors 732 communicate with RF electrodes 730 and measure the current at each electrode. A current detected by sensors 732-1 communicating with RF electrodes 730-1 is indicative of a current flowing through fat layer 706 along path 750, while a current detected by sensors 732-2 communicating with RF electrodes 730-2 is indicative of current flowing through skin layer 704 along path 752.

FIG. 7C is a cross-section view illustration of yet another exemplary embodiment in accordance with the current method and apparatus in which ultrasound transducers 720 also serve as RF electrodes as will be explained in detail below. Current sensors 736 communicating with ultrasound transducers 720 electrodes and current sensors 732 on RF electrodes 730 measure the current at each electrode. A current detected by sensors 732 communicating with RF electrodes 730 is indicative of a current flowing through fat layer 706 along path 750, while a current detected by sensors 736 communicating with the RF electrodes of transducers 720 is indicative of current flowing through skin layer 704 along path 752.

FIG. 7D, which is a plan view simplified illustration of still another exemplary embodiment in accordance with the current method and apparatus, one or more RF electrodes 730 may be attached to the emitting surface of transducer 720. RF electrodes 730 may be made of a conductive material acoustically matched (i.e., acoustically transparent) to transducer 720 or a spacer (not shown) as described hereinabove. RF electrodes 730 may be in the form of thin electrically conducting layer such as a mesh as shown in FIG. 7D having one or more current sensors 732 dispersed, for example, along mesh intersections.

In another exemplary embodiment each one or more RF electrode 730 may be made of a distinct mesh made of a conductive material, acoustically matched and attached to the emitting surface of transducer 720 or a spacer (not shown) at separate locations as shown in FIG. 7E.

In FIG. 7F, which is a plan view simplified illustration of yet another embodiment in accordance with the current method and apparatus, at least two RF electrodes 730 and 738 may be arranged concentrically around ultrasound transducer.

Alternatively, each RF electrode may be divided into one or more external segments and one or more internal segments driven at the same potential and having the current flowing through each segment measured separately to obtain differentiation between the current flowing through skin tissue and the current flowing through fat tissue.

It will be appreciated by persons skilled in the art that the electrodes depicted in FIG. 7F need not be only circular and may be of any other suitable geometrical shape such as a square, rectangle, hexagon, etc.

FIG. 7G is a cross-section view simplified illustration of still another exemplary embodiment of the current method and apparatus. FIG. 7G illustrates a mono-polar electrical configuration of an ultrasound transducer 722 that also serves as an RF electrode similar to that depicted in FIG. 7D and a single RF electrode 730 concentrically surrounding ultrasound transducer/electrode 722 in a configuration similar to that of FIG. 7F. Both transducer/electrode 722 and electrodes 730 are equipotential and connected to a return electrode 734 located elsewhere on the body. Current sensors 732 and 736 measure the current flowing through each of transducer/electrode722 and electrode 730.

A current detected by sensor 736 communicating with transducer/electrodes 722 is indicative of a current flowing through fat layer 706 along path 750, while a current detected by sensor 732 communicating with RF electrode 730 is indicative of current flowing through skin layer 704 along path 752.

FIG. 7H is a cross-section view simplified illustration of another exemplary electrical configuration of a pair of ultrasound transducer/electrode 722 and RF electrode sets in which each set includes an ultrasound transducer 722 that also serves as an RF electrode and a single RF electrode 730 concentrically surrounding ultrasound transducer/electrode 722 in a configuration similar to that of FIG. 7F.

Each pair of RF electrodes and transducer/electrodes (i.e., pair 730-1/730-2 and pair 722-1/722-2) are equipotential. The configuration may also include a separate return electrode (not shown) positioned elsewhere on the body.

Current sensors 736 communicating with ultrasound transducers/electrodes 722 and sensors 732 on RF electrodes 730 measure the current at each electrode. A current detected by sensors 736 communicating with transducers/electrodes 722 is indicative of a current flowing through fat layer 706 along path 750, while a current detected by sensors 732 communicating with RF electrodes 730 is indicative of current flowing through skin layer 704 along path 752.

In FIG. 7I, which is illustrates yet another exemplary embodiment of the current method and apparatus, RF electrode 730 is arranged concentrically about ultrasound transducer/electrode 722 of the type, for example, depicted in FIG. 7D above.

In any of the ultrasound transducer 720/722 one or more RF electrodes 730 configurations described above, transducer 720/722 and electrode 730 may abut each other, be positioned in propinquity to each other or be at a distance from each other.

It will be appreciated by persons skilled in the art that the current method and apparatus are by no means limited to the exemplary embodiments and configuration examples or combination thereof set forth hereinabove.

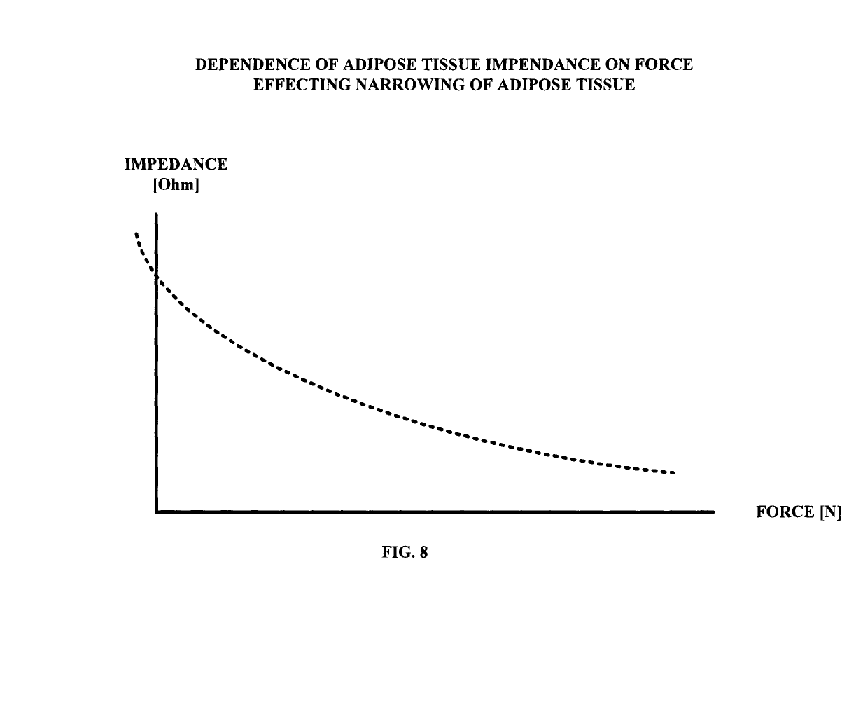

It has been found experimentally, and as shown in FIG. 8, which is a graph illustrating the dependence of adipose tissue impedance on a force effecting narrowing of the adipose tissue, that when coupling an apparatus/applicator, such as fat thickness measuring device applicator 600 (FIG. 6) or a body aesthetic treatment device applicator, to the skin, an inverse correlation exists between impedance of the tissue below the apparatus/applicator-skin contact area and the force of coupling (N) effecting narrowing of the tissue.

The physical explanation is as follows: The resistance to current flowing though skin layer 604 (FIG. 6) is constant since there is no narrowing of skin layer 604 (FIG. 6) between electrodes 630 (FIG. 6) during the application of force of coupling. On the other hand, the applied level of force of coupling effects narrowing of fat layer 606 (FIG. 6) making the effective fat layer thickness (d) smaller (i.e., narrower).

The narrowing of fat layer 606 effected by the increasing force of application (N) brings about a decrease in the resistance/impedance to current flowing along the path through fat layer 606 and through fat 606 and muscle 608.

Changes in the recorded impedance to a current flowing through tissue layers 604, 606 and 608, or in the current itself (e.g., employing current sensors), are reflective of the changes in thickness (d) or narrowing of fat layer 606.

Measuring changes in tissue impedance concurrently or intermittently with ultrasound measurement of adipose tissue layer thickness (d), employing the methods and devices described hereinabove, may provide a more accurate indication of the force of coupling (N) of fat thickness measuring device applicator 600 or of a body contouring device applicator, to the skin at any certain time.

Additionally, measuring changes in tissue impedance concurrently or intermittently with ultrasound measurement of adipose tissue layer thickness (d), employing the methods and devices described hereinabove, may also enable to extract from the thickness and impedance data one or more physical properties of the adipose tissue such as adipose tissue thickness dependence on force, adipose tissue thickness at zero force and adipose tissue electrical properties including adipose tissue conductivity and/or permittivity.

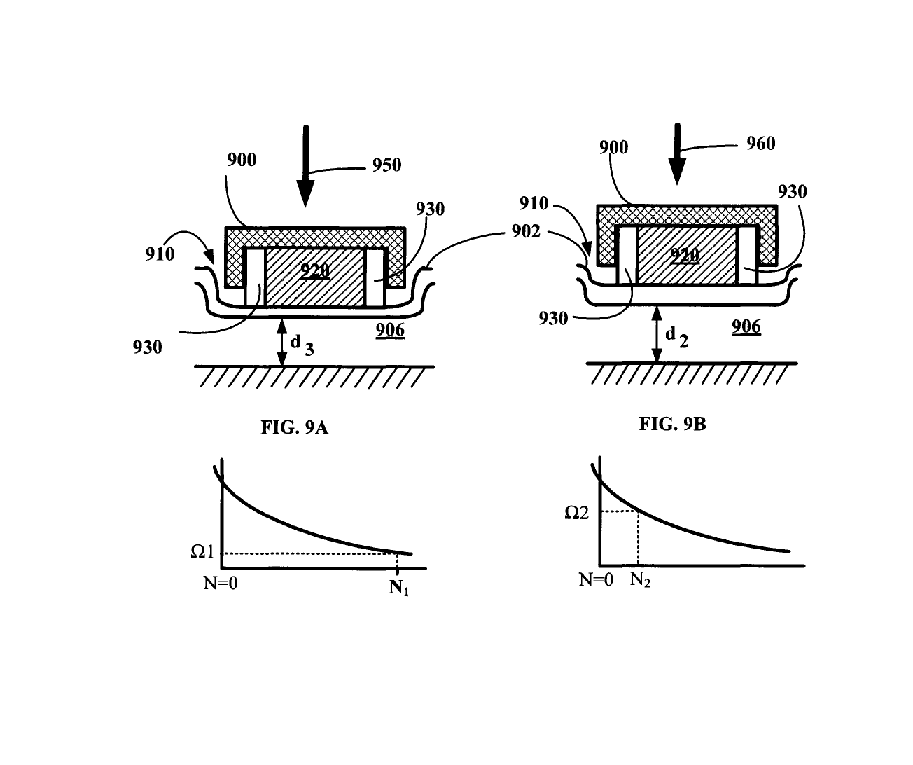

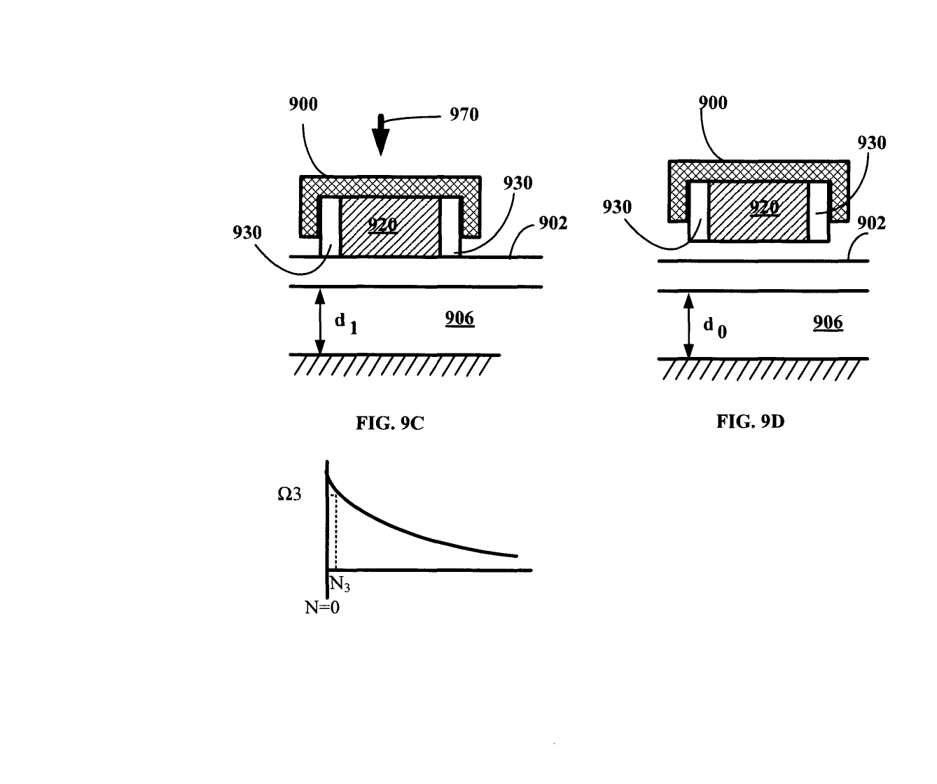

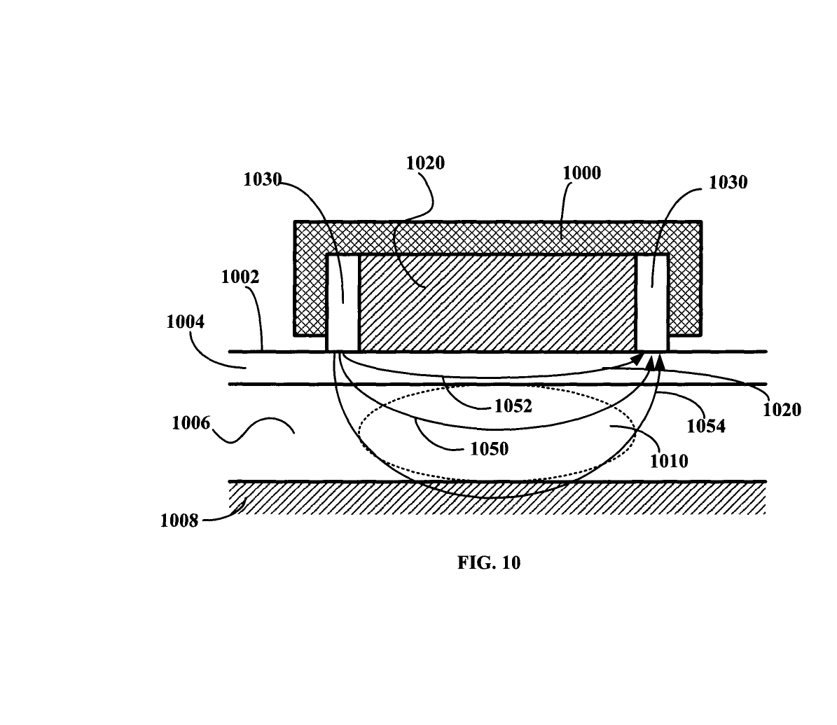

For example, applicator 600 may be coupled to surface 602 of skin 604 employing a method similar to that described in FIG. 2 hereinabove and shown in FIGS. 9A, 9B, 9C and 9D, which are simplified illustrations of another exemplary method of implementation of the embodiment of FIG. 6 in accordance with the current method and apparatus:

In FIG. 9A, applicator 900, including transducer 920 and RF electrodes 930 is coupled to the surface 902 of skin layer 904 at a certain level of force (N₁), indicated by an arrow 950, creating depression 910 and compressing fat tissue layer 906 to a thickness (d₃). Transducer 920 is activated to emit ultrasound beams into the tissue. The received reflected beam signals are then recorded. Concurrently, the impedance of adipose tissue layer 906 between RF electrodes 930 is measured, in this example, as being (Ω₁).

In FIG. 9B the level of force at which applicator 900 is coupled to surface 902 is then gradually reduced, manually or automatically, as indicated by arrow 960 to a level of force (N₂) bringing about the reduction in the depth of depression 910 of skin layer 904 and an increase in fat tissue layer 906 thickness to a thickness (d₂). Transducer 920 is activated to emit ultrasound beams into the tissue and the received reflected beam signals are then recorded. Concurrently, the impedance of adipose tissue layer 906 between RF electrodes 930 is measured and recorded, at this point in time, as being, for example, (Ω₂).

In FIG. 9C the process described in FIG. 9B is repeated. The level of force at which applicator 900 is coupled to surface 902 is then gradually reduced, manually or automatically, to a level of force (N₃) as indicated by arrow 970 up to a point of disengagement (end point or zero force point) of Transducer 920 emitting surface and of RF electrodes 930 from surface 902. At this end point, which is as close to optimal as possible, applicator 900 is coupled to surface 902 at a minimal level of force (N₃) or, optimally, with no application of force (N=0). No or barely noticeable depression 910 exists. Transducer 920 is activated to emit ultrasound beams into the tissue and the received reflected beam signals are then recorded. Concurrently, the impedance of adipose tissue layer 906 between RF electrodes 930 is measured and recorded, at this point in time, as being, for example, (Ω₃). The measured fat layer thickness (d₁) is as close as possible to true thickness (d₀) which prevails at rest (with no contact between applicator 900 and skin 904 surface 902 as shown in FIG. 9D).

Immediately following the end point of FIG. 9C, contact between applicator 900 transducer 920 and RF electrodes 930 and skin 904 surface 902 is broken, as illustrated in FIG. 9D. At this instance, the measured impedance is infinitely high due to break of electrical contact between RF electrodes 930 and skin 904 surface 902 implying that the last recorded impedance value (Ω₃) represents the most precise indicator of thickness (d₁) of adipose tissue layer 906 (i.e., thickness (d₁) at time of measurement of impedance value (Ω₃) is closest to true thickness (d₀) at a zero level of force).

It will be appreciated by those skilled in the art that the steps depicted in FIGS. 9A, 9B and 9C may occur at any point along the graph shown in FIG. 8 and that every pair of measured values (N) and (Ω) may be sampled periodically and compared to any other pair of measured values (N) and (Ω) along the graph, such as the previous or next pair of values (N) and (Ω), to monitor changes in fat tissue impedance and derive thickness of adipose tissue layer 906 while reducing the level of applicator 900 coupling force (N) to arrive at the thickness of adipose tissue layer 906 at zero level of force.

Additionally, further experimentation may enable setting up a look up table to which the measured pairs of values (N) and (Ω) may be compared to derive the level of narrowing and thickness of adipose tissue layer 906 at any certain level of applicator 900 coupling pressure (N).

The selection of measured pairs of values (N) and (Ω) to be compared may be predetermined, determined in real time or determined following the treatment session.

Reference is now made to FIG. 10, which is a simplified illustration of the effect of RF frequency on tissue conductivity or impedance of tissue layers interposed between RF electrodes in accordance with the current method and apparatus. An adipose tissue thickness measuring device applicator 1000 such as that shown in FIG. 6, includes one or more ultrasound transducers 1020 and one or more RF electrodes 1030 disposed on opposite sides of ultrasound transducer 1020. Alternatively, a separate return electrode may be employed in a mono-polar configuration.

Ultrasound transducers 1020 and one or more RF electrodes 1030 may be coupled at a certain level of force to a surface 1002 of a skin layer 1004. Alternatively and optionally, ultrasound transducer 1002 may also be operative to operate as an electrode. Additionally and optionally, ultrasound transducer 1002 may also include a spacer and operate as described in detail hereinabove.

As discussed hereinabove, electrodes 1030 placed, for example, on the surface 1002 of skin 1004 may be employed to determine the electrical impedance of the adipose tissue segment 1010 disposed between electrodes 1030 by applying a known voltage between electrodes 1030. The current flows in the tissue as explained hereinabove, along current paths indicated by arrows designated reference numeral 1050, 1052, 1054. Measuring the total current at the electrode-skin surface coupling points enables to determine the conductivity or impedance of adipose tissue segment 1010.

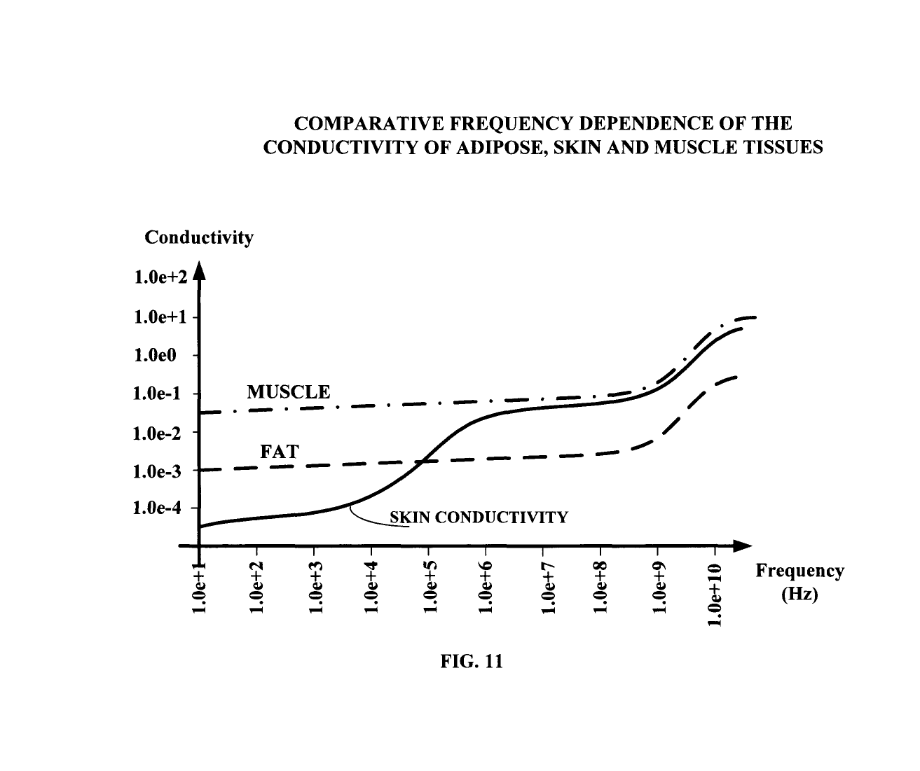

The probing current, when generated between electrodes 1030 follows the path of least impedance. As shown in FIG. 11, which is a graph illustrating the comparative frequency dependence of the conductivity of adipose, skin and muscle tissues [based on “Compilation of the Dielectric Properties of Body Tissues at RF and Microwave Frequencies”, Camelia Gabriel, PhD and Sami Gabriel, MSc., Physics Department, King's College London (http://niremf.ifac.cnr.it/docs/DIELECTRIC/Home.html)], the conductivity of adipose, skin and muscle tissues varies in accordance with the frequency of the probing current.

As illustrated in FIG. 11, at high RF frequencies, such as 100 MHz, the conductivity of wet skin is much higher than that of fat tissue allowing most of the current to flow through the skin tissue following the path indicated by reference numeral 1052 (FIG. 10). A very small flow of current will reach muscle layer 1008, following path 1054, being impeded by fat layer 1006.

At an RF frequency of approximately 50 KHz the conductivity of wet skin and adipose tissue are approximately the same allowing the probing current to flow, evenly distributed, through both tissue layers, following both paths 1050 and 1052 and also through muscle in path 1054 (FIG. 10). At frequencies below 50 KHz, the conductivity of wet skin drops dramatically below that of fat tissue allowing most of the probing current to flow through adipose tissue layer 1006 following path 1050 and a significant part through path 1054.

In accordance with the frequency dependence of the conductivity of adipose, skin and muscle tissues, when employing impedance measurement as an indicator for the level of coupling force effecting adipose tissue narrowing, such as in the exemplary method of implementation described in FIG. 9 hereinabove, the employed RF frequency is commonly in the range between 1 KHz and 1 MHz. More commonly the employed RF frequency is in the range between 5 KHz and 500 KHz and most commonly, the employed RF frequency is in the range between 10 KHz and 100 KHz.

In another embodiment, in accordance with the current method and apparatus, the measurement can be done employing several frequencies, to acquire more information on the tissue properties. One frequency may be selected from the lower end of range of frequencies, for example, about 10 kHz, to get the resistance of fat path 1050, and another frequency may be selected from at the higher end of range of frequencies, for example 1 MHz of 100 kHz to get the resistance of skin path 1052.

### Measurement of Water Content of Adipose Tissue Employing Ultrasound and RF Impedance

In yet another embodiment in accordance with the current method and apparatus, an adipose tissue thickness measuring device applicator, such as that shown in FIG. 6 may also include a mechanism operative to measure conductivity or permittivity of adipose tissue between the RF electrodes and may be employed as, for example, in the exemplary method of implementation described in FIG. 9 to provide information regarding the water content of adipose tissue.

Conductivity information may be received from the measurements of the impedance between the RF electrodes together with the adipose tissue thickness and skin thickness optionally derived from the ultrasound measurements. For example, a volume 610 (FIG. 6) of adipose tissue layer 606 may be analyzed accounting for adipose tissue layer 606 thickness (d) and known or expected conductivity values such as those shown in FIG. 11. An increase in conductivity above an expected conductivity value for the measured adipose tissue thickness (d) may indicate natural or induced infiltration of electrically conductive fluid, such as water, into the adipose tissue. The ratio between the expected conductivity value and measured conductivity value difference and the conductivity value at a measured adipose tissue layer thickness (d) may provide a quantitative indication of the water content in the tissue.

As described in FIGS. 6, 10 and 11 above, the same considerations for selecting an optimal frequency range may also be applied for obtaining the tissue water content. At a lower frequency the skin conductivity is lower, hence the impedance measured between the electrodes may be indicative of fat conductivity and therefore of the tissue water content as well. According to another embodiment measurement at more than one frequency is made to obtain data on tissue layer conductivities and calculate their water content by comparison to a known database such as a database of adipose tissue electrical properties. These measurements may be made at various forces on the applicator. The measured fat thickness together with the electrical resistance may be applied for isolating the fat dependent part of the conductivity and for obtaining a more accurate data on the water content.

In still another embodiment in accordance with the current method and apparatus and with reference to FIGS. 7A, 7B, 7C, 7E, 7F, 7G and 7H, employing internal and external electrodes driven at the same potential and measuring separately the current through each electrode enables to obtain differentiation between measurements of the current flowing through skin tissue and the current flowing through fat tissue.

It will be appreciated by persons skilled in the art that the present method and apparatus are not limited to what has been particularly shown and described hereinabove. Rather, the scope of the disclosure includes both combinations and sub-combinations of various features described hereinabove as well as modifications and variations thereof which would occur to a person skilled in the art upon reading the foregoing description and which are not in the prior art.

1.-65. (canceled)

66. A method for measuring adipose tissue physical properties comprising:
coupling to a segment of skin overlaying said adipose tissue, at a certain level of force, an ultrasound transducer having at least one resilient spacer;
emitting at least one ultrasound beam through said spacer into said segment of skin;
receiving at least one signal of a portion of said beam reflected from a spacer-skin interface;
receiving at least one signal of a portion of said beam reflected from skin-adipose tissue interface;
receiving at least one signal of a portion of said beam reflected from a spacer-skin interface;
extracting from at least two of said received signals at least one of a group of thicknesses consisting of the spacer thickness, the skin thickness and the thickness of said adipose tissue layer; and
employing at least one of the thicknesses to derive the level of force.

67. The method according to claim 66, wherein physical properties of said spacer are derived from at least one of a group consisting of selected material properties and a calibration process.

68. The method according to claim 66, wherein difference between the acoustical impedances of the spacer and the skin is selected to have the lowest values enabling detection of the reflection from the spacer-skin interface.

69. The method according to claim 66, wherein also changing said level of force manually or automatically.

70. The method according to claim 66, wherein said ultrasound beam is emitted in pulse mode.

71. The method according to claim 66, wherein also emitting said ultrasound beam and varying the frequency of said beam within a band, transforming the results from frequency domain to time domain to isolate a virtual pulse reflected from the tissue layers interfaces.

72. The method according to claim 66, further comprising;
coupling at least one RF electrode to said segment of skin and at least one electrode to said segment or any other segment of skin; and
measuring the electrical impedance between these electrodes.

73. The method according to claim 72, wherein measuring said impedance comprises employing at least one electrode having an internal segment and an external segment driven at the same potential; and
measuring separately current flowing through each electrode to obtain differentiation between the current flowing through skin tissue and the current flowing through fat tissue.

74. The method according to claim 72, wherein also comparing the measured impedance of the adipose tissue with a database of adipose tissue impedance values selected from a group of databases consisting of literature based databases and a database extracted from previous measurement extracting from said comparison the water content of said adipose tissue.

75. An apparatus for measuring physical properties of adipose tissue comprising:
an applicator housing:
at least one ultrasound transducer;
at least one resilient spacer attached to said transducer; and
a controller operative to control ultrasound beams emitted by an ultrasound transducer and analyze signals of ultrasound beams reflected from at least two of a group of interfaces consisting of a spacer-skin interface, skin-adipose tissue interface and an adipose tissue-muscle interface received by said transducer; and
wherein the controller is operative to extract from said received signals the adipose tissue layer thickness and the level of force at which said applicator is applied to the tissue.

76. The apparatus according to claim 75, wherein said resilient spacer is made of material selected from a group consisting of rubber, epoxy, and a polymer.

77. The apparatus according to claim 75, wherein said spacer is made of a resilient structure including a bias element and filled with liquid acoustic transmission media.

78. The apparatus according to claim 75, wherein physical properties of said spacer are derived from at least one of a group consisting of selected material properties and a calibration process.

79. The apparatus according to claim 75, said spacer is of thickness and acoustic velocity operative to delay beam portion reflections to a point in time beyond transmitted signal decay time.

80. The apparatus according to claim 75, further comprising including at least two RF electrodes connected to an RF voltage source, sensors operative to measure the current between the electrodes from at least one electrode, and a controller operative to calculate the electrical impedance between said electrodes.

81. The apparatus according to claim 75, wherein said at least one electrode also comprises internal and external electrode segments driven at the same potential and measuring separately current flowing through each electrode segment.

82. The apparatus according to claim 75, wherein the controller is operative to calculate at least one of fat layer thickness at zero force, fat layer thickness and force, fat layer conductivity, fat layer permittivity and water content of the fat layer.

83. The apparatus according to claim 75, wherein at least one of said RF electrodes is located at least partially on the emitting surface of said spacer.

84. The apparatus according to claim 83, wherein said RF electrodes are made of a electrically conductive material acoustically transparent to emitted ultrasound beams.

85. The apparatus according to claim 75, wherein acoustical impedance of said spacer is selected to be as close as possible to, but different than, that of skin so that to sufficiently allow detection of a reflection from spacer-skin interface.

86. A method for adipose tissue thickness measuring employing ultrasound, said method comprising:
coupling to a segment of skin overlaying said adipose tissue an ultrasound transducer at a certain level of force;
emitting consecutively at least two ultrasound beam emissions into at least said adipose tissue;
receiving signals of reflections of said ultrasound beam emissions;
recording data from said received emission signals;
gradually reducing said level of force until no emission signals are received; and
extracting data from the last received ultrasound beam emission signal indicating the thickness of said adipose tissue at a zero level of force.

87. The method according to claim 86, wherein said ultrasound emission is in pulse form.

88. The method according to claim 86, wherein also emitting said ultrasound beam and varying the frequency of said beam within a band, transforming the results from frequency domain to time domain to isolate a virtual pulse reflected from the tissue layers interfaces.

89. The method according to claim 86, wherein said emitted ultrasound beam is in the frequency range between 200 kHz and 2 MHz.

90. The method according to claim 86, wherein reducing said level of force manually or automatically.
